(19) United States
(12) Patent Application Publication
(10) Pub. No.: US 2015/0105600 A1
(43) Pub. Date: Apr. 16, 2015

(54) ADSORBENTS FOR THE SEPARATION OF PARA-XYLENE FROM C8 ALKYL AROMATIC HYDROCARBON MIXTURES, METHODS FOR SEPARATING PARA-XYLENE USING THE ADSORBENTS AND METHODS FOR MAKING THE ADSORBENTS

(71) Applicant: UOP LLC, Des Plaines, IL (US)

(72) Inventors: Patrick C. Whitchurch, Sleepy Hollow, IL (US); Santi Kulprathipanja, Inverness, IL (US); Gregory F. Maher, Aurora, IL (US)

(73) Assignee: UOP LLC, Des Plaines, IL (US)

(21) Appl. No.: 14/054,687

(22) Filed: Oct. 15, 2013

(51) Int. Cl. C07C 7/12; B01J 20/30; B01J 20/18

(52) U.S. Cl. CPC C07C 7/12; B01J 20/18; B01J 20/3071
USPC 585/828; 423/700; 423/713

(57) ABSTRACT

Embodiments of adsorbents for separating para-xylene from a mixture of C₈ alkyl aromatic hydrocarbons, methods for making such adsorbents, and methods for separating para-xylene using such adsorbents are provided. In one example, an adsorbent comprises a binderless adsorbent. The binderless adsorbent comprises zeolite X and has a K₂O/(K₂O+BaO+Na₂O) molar ratio of from about 0.15 to about 0.4.

### TECHNICAL FIELD

The technical field relates generally to adsorbents and methods for separating para-xylene from a mixture of C₈ alkyl aromatic hydrocarbons, and more particularly relates to binderless adsorbents comprising zeolite X and having improved para-xylene adsorption capacity, methods for making such binderless adsorbents, and methods for separating para-xylene from a mixture of C₈ alkyl aromatic hydrocarbons using such binderless adsorbents.

### BACKGROUND

Alkylated aromatics include among other compounds the various isomers of xylene, i.e., ortho-, meta-, and para-xylene. Of these, para-xylene is of particular value as a large volume chemical for the production of polyethylene terephthalate (PET), which is used, for example, as polyester fiber, film, and resin for a variety of applications. Because of downstream demand, the para-xylene market is robust and generally sees steady year-to-year demand growth.

Major sources of para-xylene include mixed xylene streams produced from the refining of crude oil. Examples of such streams include those produced from commercial xylene isomerization processes and from the separation of C₈ alkyl aromatic hydrocarbons fractions derived from a catalytic reformate by liquid-liquid extraction and fractional distillation. Typically, para-xylene is recovered from these mixed xylene streams by adsorptive separation. In one example, a feed stream containing a mixture of C₈ alkyl aromatic hydrocarbons (e.g., ortho-xylene, meta-xylene, para-xylene, ethyl benzene, and the like) is contacted with adsorbent particles, each containing an adsorbent material held together with a binder (e.g., clay), and para-xylene from the feed stream is adsorbed onto the adsorbent particles. The adsorbent particles are subsequently contacted with para-diethyl benzene (p-DEB) to desorb para-xylene from the adsorbent particles and form an extract stream containing the p-DEB and para-xylene. Para-xylene is then recovered from the extract stream, for example, by fractionation and the p-DEB can be recycled for desorbing additional para-xylene. Unfortunately, this approach has several challenges. First, further improvements in para-xylene adsorption capacity over conventional adsorbent particles are needed to meet the growing demand for para-xylene. Second, desorbing para-xylene from the adsorbent particles with p-DEB can also desorb other heavier aromatic hydrocarbons, e.g., C₉⁺ aromatic hydrocarbons such as triethylbenzene and diethyl benzene, from the adsorbent particles and these heavier aromatic hydrocarbons can build up over time in the p-DEB, requiring additional fresh makeup p-DEB and reducing overall process efficiency.

Accordingly, it is desirable to provide adsorbents having improved para-xylene adsorption capacity, methods for making such adsorbents, and methods for separating para-xylene from a mixture of C₈ alkyl aromatic hydrocarbons using such adsorbents. Moreover, it is desirable to provide adsorbents that help improve overall process efficiency for recovering para-xylene, methods for making such adsorbents, and methods for separating para-xylene from a mixture of C₈ alkyl aromatic hydrocarbons using such adsorbents. Furthermore, other desirable features and characteristics of the present invention will become apparent from the subsequent detailed description and the appended claims, taken in conjunction with the accompanying drawings and this background.

### BRIEF SUMMARY

Adsorbents for separating para-xylene from a mixture of C₈ alkyl aromatic hydrocarbons, methods for making such adsorbents, and methods for separating para-xylene using such adsorbents are provided herein. In accordance with an exemplary embodiment, an adsorbent comprises a binderless adsorbent. The binderless adsorbent comprises zeolite X and has a K₂O/(K₂O+BaO+Na₂O) molar ratio of from about 0.15 to about 0.4.

In accordance with another exemplary embodiment, a method for separating para-xylene from a mixture of C₈ alkyl aromatic hydrocarbons is provided. The method comprises the steps of contacting a binderless adsorbent with a feed stream comprising the mixture of C₈ alkyl aromatic hydrocarbons to adsorb para-xylene from the feed stream and form a para-xylene-adsorbed binderless adsorbent. The binderless adsorbent comprises zeolite X and has a K₂O/(K₂O+BaO+Na₂O) molar ratio of from about 0.15 to about 0.4. Para-xylene is desorbed from the para-xylene-adsorbed binderless adsorbent.

In accordance with another exemplary embodiment, a method for making an adsorbent for separating para-xylene from a mixture of C₈ alkyl aromatic hydrocarbons is provided. The method comprises the steps of forming a particle comprising a zeolite X precursor and a first portion of zeolite X. The zeolite X precursor of the particle is activated at activation conditions effective to form an activated zeolite X precursor. The particle is digested with a caustic solution to convert the activated zeolite X precursor to a second portion of zeolite X and form a binderless zeolite X particle having ion exchangeable sites with cations. The cations are exchanged with barium and potassium ions at the ion exchangeable sites to form a binderless adsorbent. The binderless adsorbent comprises the zeolite X and has a K₂O/(K₂O+BaO+Na₂O) molar ratio of from about 0.15 to about 0.4.

### BRIEF DESCRIPTION OF THE DRAWINGS

The various embodiments will hereinafter be described in conjunction with the following drawing figures, wherein like numerals denote like elements, and wherein:

FIG. 1 is a block diagram of a method for making an adsorbent for separating para-xylene from a mixture of C₈ alkyl aromatic hydrocarbons in accordance with an exemplary embodiment; and

FIG. 2 is a schematic illustration of an apparatus and a method for separating para-xylene from a mixture of C₈ alkyl aromatic hydrocarbons in accordance with an exemplary embodiment.

### DETAILED DESCRIPTION

The following Detailed Description is merely exemplary in nature and is not intended to limit the various embodiments or the application and uses thereof. Furthermore, there is no intention to be bound by any theory presented in the preceding background or the following detailed description.

Various embodiments contemplated herein relate to adsorbents for separating para-xylene from a mixture of C₈ alkyl aromatic hydrocarbons, methods for making such adsorbents, and methods for separating para-xylene using such adsorbents. As used herein, Cₓ means hydrocarbon molecules that have “X” number of carbon atoms, Cₓ⁺ means hydrocarbon molecules that have “X” and/or more than “X” number of carbon atoms, and Cₓ⁻ means hydrocarbon molecules that have “X” and/or less than “X” number of carbon atoms.

The exemplary embodiments taught herein provide an adsorbent that is binderless (binderless adsorbent) and comprises zeolite X. Zeolites are crystalline aluminosilicate compositions that are microporous and that are formed from corner sharing AlO₂ and SiO₂ tetrahedra. Synthetic zeolites are prepared via hydrothermal synthesis employing suitable sources of Si, Al and structure directing agents or templates such as alkali metals, alkaline earth metals, amines, or organoammonium cations. The structure directing agents reside in the pores of the zeolite and are largely responsible for the particular structure that is ultimately formed. These species balance the framework charge associated with aluminum and can also serve as space fillers. Zeolites are characterized by having pore openings of uniform dimensions, having a significant ion exchange capacity, and being capable of reversibly desorbing an adsorbed phase that is dispersed throughout the internal voids of the crystal without significantly displacing any atoms that make up the permanent zeolite crystal structure. The crystalline structure of zeolite X is well known and described in detail in U.S. Pat. No. 2,882,244 and in “Atlas of Zeolite Structure Types”, W. M. Meier, D. H. Olson and C. Baerlocher, 5^{th} revised edition, 2001, Elsevier. As will be discussed in further detail below, the binderless adsorbent is composed substantially of zeolite X and is substantially absent of any non-zeolitic or amorphous materials, such as, for example, clays or other conventional binders.

In an exemplary embodiment, preparation of the binderless adsorbent includes exchanging cations (e.g., Na+) at ion exchangeable sites in the zeolite X with barium ions (Ba+) and potassium ions (K+) to form the binderless adsorbent having a molar ratio of potassium oxide (K₂O) to potassium oxide plus barium oxide (BaO) plus sodium oxide (Na₂O) (hereinafter “K₂O/(K₂O+BaO+Na₂O) molar ratio”) of from about 0.15 to about 0.4. It has been found that eliminating or substantially eliminating conventional binders, which normally contribute only non-selective pore volume, can significantly increase adsorbent capacity of the adsorbent for para-xylene and further, for a desorbent such as toluene for desorbing para-xylene from the binderless adsorbent. Additionally, it has been found that heavier aromatic hydrocarbons such as C₉⁺ aromatic hydrocarbons, for example triethylbenzene and diethyl benzene, do not tend to build up over time in toluene relative to p-DEB when used in an adsorptive separation process. Furthermore, the relatively high K₂O/(K₂O+BaO+Na₂O) molar ratio of the binderless adsorbent effectively reduces the quantity of toluene required to desorbent a given amount of para-xylene from the binderless adsorbent. As such, the binderless adsorbent allows for the practical use of toluene as a desorbent and helps to improve the overall adsorptive separation process efficiency for para-xylene.

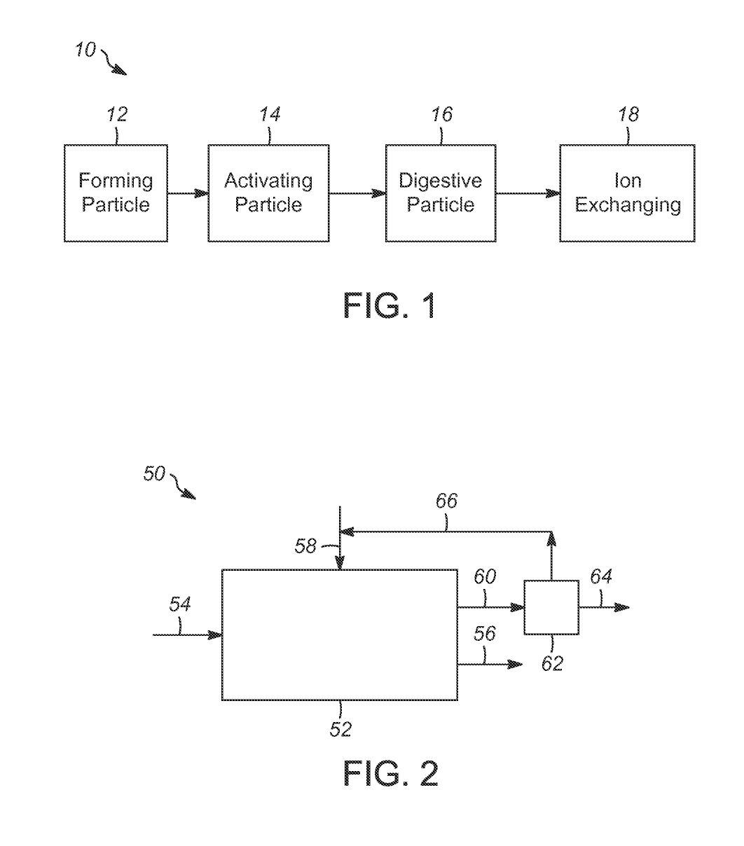

FIG. 1 is a block diagram of a method 10 for making an adsorbent for separating para-xylene from a mixture of C₈ alkyl aromatic hydrocarbons in accordance with an exemplary embodiment. The method 10 includes forming a particle(s) (step 12) of a prepared (or already made) portion of zeolite X and a zeolite X precursor.

In an exemplary embodiment, the prepared portion of the zeolite X has a small-crystal-size, such as from about 1 to about 3 μm. In an exemplary embodiment, the small-crystallite-size zeolite X is prepared from a seeded synthesis, where a seed or initiator material, used as a means of nucleation or starting zeolite crystallite growth, is first prepared and then blended into a gel composition at a gel composition to seed ratio corresponding to a targeted crystallite size. The gel composition to seed ratio governs the relative number or concentration of nucleation sites, which in turn affects the crystallite size of the zeolite X that is synthesized. Higher amounts or concentrations of seed directionally reduce the crystallite size. For example, zeolite X preparations having average crystallite sizes of 2 μm and 0.5 μm can be made using gel to seed ratios of about 5400:1 and 85:1, by weight, respectively. The gel to seed ratios can be varied to achieve other average crystallite sizes as desired. A typical gel composition comprises Na₂O, SiO₂, Al₂O₃, and water. For each mole of Al₂O₃, from about 1 to about 5 moles of Na₂O and SiO₂, and from about 100 to about 500 moles of water, can be used in the gel.

The gel composition may be prepared by combining a gel makeup solution with an aluminate makeup solution containing, for example, about 12 weight % (wt. %) of alumina. The gel makeup solution is prepared by mixing water, a caustic solution, and sodium silicate, and cooling the mixture to about 38° C. The aluminate makeup solution is prepared by dissolving alumina trihydrate in the caustic solution, with heating as needed for dissolution, followed by cooling and aging at about 38° C. prior to combining it with the gel makeup solution. The gel makeup solution and aluminate solution are then combined under vigorous agitation for a short period (e.g., about 30 minutes), prior to adding a predetermined amount of a seed.

In an exemplary embodiment, the seed is prepared in a similar manner to the gel composition. A seed composition also comprises Na₂O, SiO₂, Al₂O₃, and water. For each mole of Al₂O₃, from about 10 to about 20 moles of Na₂O and SiO₂, and from about 150 to about 500 moles of water, can be used. The aluminate solution used in preparing the seed may contain, for example, about 18 wt. % of alumina. After the gel composition and seed are combined, the mixture is heated while agitation is maintained, and then aged under agitated conditions for a time of from about 5 to about 50 hours and at a temperature of from about 25 to about 300° C. to achieve the desired crystallite formation from the seed nuclei. The resulting solid material may then by filtered, washed, and dried to obtain the prepared, small-crystallite-size zeolite X.

In another exemplary embodiment, the prepared portion of the zeolite X has a nano-crystal-size, such as from about 1 to about 500 nm. In an exemplary embodiment, synthesis of the nano-size zeolite includes an initiator. The initiator is a concentrated, high pH aluminosilicate solution and has a composition represented by an empirical formula of:

Al₂O₃:aSiO₂:bM_{2/m}O:cH₂O

where “a” has a value of from about 4 to about 30, “b” has a value of from about 4 to about 30, and “c” has a value of from about 50 to about 500, “m” is the valence of M and has a value of +1 or +2 and M is a metal selected from the group consisting of alkali metals, alkaline earth metals and mixtures thereof, for example lithium, sodium, potassium and mixtures thereof. The initiator is obtained by mixing reactive sources of Al, Si and M plus water.

The aluminum sources include but are not limited to, aluminum alkoxides, precipitated alumina, aluminum hydroxide, aluminum salts and aluminum metal. Specific examples of aluminum alkoxides include, but are not limited to aluminum orthosec-butoxide, and aluminum orthoisopropoxide. Sources of silica include but are not limited to tetraethylorthosilicate, fumed silicas, precipitated silicas and colloidal silica. Sources of the M metals include but are not limited to the halide salts, nitrate salts, acetate salts, and hydroxides of the respective alkali or alkaline earth metals. When M is sodium, the sources are, for example, sodium aluminate and/or sodium silicate. The sodium aluminate is synthesized in situ by combining gibbsite with sodium hydroxide. Once the initiator is formed it is aged at a temperature of about 0 to about 100° C. for a time sufficient for the initiator to exhibit the Tyndall effect. Usually the time varies from about 1 hour to about 14 days, for example from about 12 hours to about 10 days.

In an exemplary embodiment, synthesis of the nano-size zeolite also includes a reaction solution. The reaction solution has a composition represented by an empirical formula of:

Al₂O₃:dSiO₂:eM_{2/m}O:fR_{2/p}O:gH₂O

where “d” has a value of from about 4 to about 30, “e” has a value of from about 4 to about 30, “f” has a value of from 0 to about 30 and “g” has a value of from about 5 to about 500, “p” is the valence of R and has a value of +1 or +2, R is an organoammonium cation selected from the group consisting of quaternary ammonium ions, protonated amines, protonated diamines, protonated alkanolamines, diquaternary ammonium ions, quaternized alkanolamines and mixtures thereof. The reaction solution is formed by combining reactive sources of Al, Si, M and R plus water. The sources of aluminum, silicon and M are as described above, while the sources of R include but are not limited to hydroxide, chloride, bromide, iodide and fluoride compounds. Specific examples include without limitation ethyltrimethylammonium hydroxide (ETMAOH), diethyldimethylammonium hydroxide (DEDMAOH), propylethyldimethylammonium hydroxide (PEDMAOH), trimethylpropylammonium hydroxide, trimethylbutylammonium hydroxide (TMBAOH), tetraethylammonium hydroxide, hexamethonium bromide, tetramethylammonium chloride, N,N,N,N′,N′,N′-hexamethyl 1,4 butanediammonium hydroxide and methyltriethylammonium hydroxide. The source of R may also be neutral amines, diamines, and alkanolamines, such as, for example, triethanolamine, triethylamine, and N,N,N′,N′tetramethyl-1,6-hexanediamine

A reaction mixture is now formed by mixing the initiator and reaction solution. In an exemplary embodiment, the initiator is slowly added to the reaction solution and stirred for an additional period of time to ensure homogeneity. The resultant reaction mixture is now charged to an autoclave and reacted under autogenous pressure at a temperature of from about 25 to about 200° C. for a time of from about 1 hr to about 40 days. The reaction can be carried out either with or without stirring. After the reaction is complete, a solid zeolite (zeolite X) is separated from the reaction mixture by means well known in the art such as by filtration or centrifugation, and is washed with deionized water and dried in air at ambient temperature (e.g., about 20 to 25° C.) up to about 100° C. The exchangeable cations M and R can be exchanged for other desired cations and in the case of R can be removed by heating to provide the hydrogen form of the nano-size zeolite.

As discussed above, the nano-size zeolite X, small-crystallite-size zeolite X, or a relatively larger-crystallite-size zeolite X (e.g., conventional zeolite X having a crystal-size of from about 3 to about 100 μm) may then be used in the synthesis of the binderless adsorbent by combining this “prepared” or already made portion with the zeolite X precursor (step 12). Zeolite X precursors include clays such as kaolin, kaolinites, and halloysite, and other minerals such as hydrotalcites, and solid silica and alumina sources such as precipitated and fumed amorphous silica, precipitated alumina, gibbsite, boemite, bayerite, and transition aluminas such gamma and eta alumina, and zeolite seed solutions and suspensions obtained from sodium silicate and sodium aluminate and similar reagents, which can be formed in an intimate mixture with the crystallites of the prepared portion of zeolite X. The forming procedure involves combining the zeolite X precursor, exemplified by kaolin clay, with the zeolite X powder of the prepared portion of zeolite X and optionally other additives such as pore generating materials (e.g., corn starch to provide macroporosity) and water as needed to obtain the proper consistency for shaping. Shaping or forming into beads, spheres, pellets, and/or the like, can be performed using conventional methods including bead forming processes such as Nauta mixing, tumbling, or drum rolling to prepare larger particles (e.g., in the range of about 16-60 Standard U.S. Mesh size).

In an exemplary embodiment, the formed particles comprising the prepared portion of zeolite X and the zeolite X precursor are then activated (step 14) at a temperature of from about 500 to about 700° C. In the embodiment, the zeolite X precursor comprises kaolin clay and activation causes this material to undergo endothermic dehydroxylation, whereby the disordered, meta-kaolin phase is formed.

Following activation, caustic digestion of the formed particles (step 16), using for example sodium hydroxide, converts the activated zeolite X precursor into zeolite X, resulting in binderless zeolite X particles that may comprise or consist essentially of zeolite X having an average crystallite size associated with (i) conventional zeolite X, (ii) small-crystallite-size zeolite X, or (iii) nano-size zeolite X, as discussed above. Otherwise, the binderless adsorbent may comprise or consist essentially of the prepared portion of zeolite X having any of these average crystallite sizes associated with (i), (ii), or (iii) in combination with the converted portion of zeolite X having any other of these average crystallite sizes. In an exemplary embodiment, the prepared portion of the zeolite X has an average crystalline size of from about 1 to about 3 μm and the converted portion of the zeolite X is nano-size zeolite X having an average crystalline size of from about 1 to about 500 nm.

The silica to alumina molar ratio of the converted portion of zeolite X, as well as the contribution of this material in the final adsorbent formulation, may be varied according to the type and amount of zeolite X precursor that is incorporated into the formed particles. Typically, the silica to alumina ratio of the zeolite X precursor is substantially conserved upon conversion into zeolite X. In an exemplary embodiment, the silica to alumina molar ratio of the prepared and converted portions of zeolite X is from about 2 to about 2.6. In one example, a typical kaolin clay has a silica to alumina molar ratio from about 2 to about 2.2 and the converted portion of zeolite X has a silica to alumina ratio of from about 2 to about 2.2. In another example, the prepared and converted portions have differing silica to alumina ratios of from about 2.4 to about 2.6 and from about 2 to about 2.2, respectively.

In an exemplary embodiment, non-zeolitic material is substantially absent in the binderless zeolite X particles (e.g., non-zeolitic material present in the binderless zeolite X particles in an amount of less than about 2 wt. %, such as less than about 1 wt. %, such as less than 0.5 wt. %, for example about 0 wt. %). The absence or substantial absence of non-zeolitic or amorphous material may be confirmed by analysis of the binderless zeolite X particles using X-ray diffraction and/or high resolution scanning electron microscopy (HR-SEM) to verify crystal structure.

In an exemplary embodiment, the prepared and converted portions of zeolite X of the binderless zeolite X particles have ion exchangeable sites with cations (e.g., sodium ions) that may be partially or wholly exchanged (step 18) with barium and potassium ions using known techniques to form a binderless adsorbent. In one example, the binderless adsorbent, synthesized with zeolite X having at least some of its ion exchangeable sites in sodium ion form, is immersed in a solution containing barium and potassium ions for a time of from about 0.5 to about 10 hours and at a temperature of from about 20 to about 125° C. to affect ion exchange or replacement of sodium ions with barium and potassium ions. Ion exchange can also be conducted in a column operation according to known techniques, for example by pumping a preheated barium chloride/potassium chloride solution(s) through a column of the binderless zeolite X particles to completely displace the sodium cations of the prepared and converted portions of zeolite X. Filtration of the binderless adsorbent, removal from the solution, and re-immersion in a fresh solution (e.g., having the same or different ratios or cations or other types of cations) can be repeated until a desired level of exchange, with the desired types and ratios of cations, is achieved. In an exemplary embodiment, the binderless adsorbent has at least about 95% or substantially all (e.g., at least about 99%) of the zeolite X ion exchangeable sites exchanged with a combination of barium and potassium. In an exemplary embodiment, the binderless adsorbent has a K₂O/(K₂O+BaO+Na₂O) molar ratio of from about 0.15 to about 0.4, for example from about 0.2 to about 0.35, a BaO/(K₂O+BaO+Na₂O) molar ratio of from about 0.6 to about 0.85, for example from about 0.65 to about 0.8, and a Na₂O/(K₂O+BaO+Na₂O) molar ratio of from about 0.001 to about 0.04. In an exemplary embodiment, the binderless adsorbent comprises the prepared portion of zeolite X present in an amount of from about 60 to about 95 wt. % of binderless adsorbent and the converted portion of zeolite X present in an amount of from about 5 to about 40 wt. % of the binderless adsorbent.

One consideration associated with the overall adsorbent performance is the water content of the binderless adsorbent. The water content may be determined by a Loss of Ignition (LOI) test that measures the weight difference between an initial weight of a sample of binderless adsorbent at ambient conditions and a final weight of the sample after drying at about 900° C. under an inert gas purge such as nitrogen for a period of time, such as about 2 hours, to achieve a constant weight. In an exemplary embodiment, the binderless adsorbent has a LOI or water content of from about 2 to about 5 wt. %. Other methods known to those skilled in the art may also be used for determining the water content of the binderless absorbed.

FIG. 2 is a schematic illustration of an apparatus 50 for separating para-xylene from a mixture of C₈ alkyl aromatic hydrocarbons in accordance with an exemplary embodiment. The apparatus 50 comprises an adsorption zone 52 that contains the binderless adsorbent as discussed above. As used herein, the term “zone” refers to an area including one or more equipment items and/or one or more sub-zones. Equipment items can include one or more adsorbers, adsorber beds, and/or adsorber vessels, reactors, regenerators, heaters, exchangers, coolers/chillers, pipes, pumps, compressors, and controllers. Additionally, an equipment item, such as an adsorber, reactor, dryer, or vessel, can further include one or more zones or sub-zones. In an exemplary embodiment, the adsorption zone 52 is configured as a simulated moving bed as is known in the art. Alternatively, the absorption zone 52 may be configured in any other configuration for adsorption separation known to those skilled in the art.

As illustrated, a feed stream 54 is introduced to the adsorption zone 52. In an exemplary embodiment, the feed stream 54 comprises a mixture of C₈ alkyl aromatic hydrocarbons such as ortho-xylene, meta-xylene, para-xylene, ethyl benzene, and the like as well as possibly some heavier aromatic hydrocarbons such as C₉⁺ aromatic hydrocarbons, for example triethylbenzene and diethyl benzene. The feed stream 54 is advanced in the adsorption zone 52 and contacts the binderless adsorbent at adsorption conditions effective to selectively adsorb, in an adsorbed phase, para-xylene in preference to ortho-xylene, meta-xylene, ethyl benzene, and/or the heavier aromatic hydrocarbons. These other C₈ alkyl aromatic hydrocarbons and C₉⁺ aromatic hydrocarbons of the feed stream 54 are passed then through the adsorption zone 52 as a non-adsorbed phase and exit as a raffinate stream 56, leaving a para-xylene-adsorbed binderless adsorbent in the adsorption zone 52. In an exemplary embodiment, the adsorption conditions include a temperature of from about 100 to about 160° C., such as from about 125 to about 155° C., for example from about 130 about 140° C.

In an exemplary embodiment, a desorbent stream 58 is introduced to the adsorption zone 52. The desorbent stream 58 comprises a desorbent which is generally any material capable of desorbing an extract component from the binderless adsorbent. In an exemplary embodiment, the desorbent is toluene. The desorbent stream 58 is advanced in the adsorption zone 52 and contacts the para-xylene-adsorbed binderless adsorbent to desorb para-xylene to regenerate the binderless adsorbent and form an extract stream 60 that contains the desorbent and para-xylene. In an exemplary embodiment, the extract stream 60 is passed along to a recovery/fractionation zone 62 to separate para-xylene from the extract stream 60 and form a para-xylene product stream 64 and a recycle desorbent stream 66. As illustrated, the recycle desorbent stream 66 is recycled back to the adsorption zone 52.

### EXAMPLES

The following are examples (shown in the table) of various samples of binderless adsorbent (designated as Binderless Zeolite X Adsorbent) in accordance with exemplary embodiments compared to a conventional zeolite 13 X adsorbent with binder (designated as Zeolite 13X Adsorbent w/ Binder (Baseline)). The examples are provided for illustration purposes only, and are not meant to limit the various embodiments of the present invention in any way.

TABLE

| | | P-X Cap | | | |
|---|---|---|---|---|---|
| | LOI, wt. | Relative | BaO/ | K2O/ | Na₂O/ |
| | % (Water | Increase to | (K2O + BaO + Na2O) | (K2O + BaO + Na2O) | (K2O + BaO + Na2O) |
| Adsorbent | content) | Baseline | molar ratio | molar ratio | molar ratio |
| Zeolite 13X | 3.05 | 0% | 0.69 | 0.28 | 0.02 |
| Adsorbent w/Binder | | | | | |
| (Baseline) | | | | | |
| Binderless Zeolite X | 3.23 | 10% | 0.835 | 0.159 | 0.005 |
| Adsorbent #1, Test | | | | | |
| #1 | | | | | |
| Binderless Zeolite X | 3.23 | 12% | 0.835 | 0.159 | 0.005 |
| Adsorbent #1, Test | | | | | |
| #2 | | | | | |
| Binderless Zeolite X | 2.97 | 20% | 0.8 | 0.2 | 0.004 |
| Adsorbent #2 | | | | | |
| Binderless Zeolite X | 3.1 | 23% | 0.71 | 0.29 | 0.003 |
| Adsorbent #3, Test | | | | | |
| #1 | | | | | |
| Binderless Zeolite X | 3.1 | 23% | 0.71 | 0.29 | 0.003 |
| Adsorbent #3, Test | | | | | |
| #2 | | | | | |
| Binderless Zeolite X | 2.97 | 26% | 0.67 | 0.33 | 0.004 |
| Adsorbent #4 | | | | | |
| Binderless Zeolite X | 2.94 | 21% | 0.66 | 0.34 | 0.003 |
| Adsorbent #5 | | | | | |
| Binderless Zeolite X | 3.05 | 9% | 0.614 | 0.383 | 0.003 |
| Adsorbent #6 | | | | | |

The binderless zeolite X adsorbents #1-#6 were prepared by ion exchanging various binderless X zeolite beads at about 90° C. with a solution containing potassium chloride and barium chloride. The binderless zeolite X adsorbents were then dried at about 290° C. The dried binderless zeolite X adsorbents were contacted with a feed stream containing nonane (as a tracer) present in an amount of about 5.5 wt. %, toluene present in an amount of about 25.1 wt. %, para-xylene present in an amount of about 14.8 wt. %, ethyl benzene present in an amount of about 10 wt. %, and ortho-xylene present in an amount of about 44.4 wt. %. The adsorbents were each contacted by the feed stream at a temperature of about 150° C. The results were compared to a baseline (conventional) zeolite 13X adsorbent with binder. As illustrated, the para-xylene adsorption capacity (P-X Cap) relative to the baseline showed increases of from about 9 to about 26% over the baseline for the various binderless zeolite X adsorbents #1-#6.

Accordingly, adsorbents for separating para-xylene from a mixture of C₈ alkyl aromatic hydrocarbons, methods for making such adsorbents, and methods for separating para-xylene using such adsorbents have been described. The exemplary embodiments taught herein provide an adsorbent that comprises a binderless adsorbent. The binderless adsorbent comprises zeolite X and has a K₂O/(K₂O+BaO+Na₂O) molar ratio of from about 0.15 to about 0.4.

While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing an exemplary embodiment of the disclosure. It being understood that various changes may be made in the function and arrangement of elements described in an exemplary embodiment without departing from the scope of the disclosure as set forth in the appended claims.

What is claimed is:

1. An adsorbent for separating para-xylene from a mixture of C₈ alkyl aromatic hydrocarbons, the adsorbent comprising:
a binderless adsorbent comprising zeolite X and having a K₂O/(K₂O+BaO+Na₂O) molar ratio of from about 0.15 to about 0.4.

2. The adsorbent of claim 1, wherein the K₂O/(K₂O+BaO+Na₂O) molar ratio is from about 0.2 to about 0.35.

3. The adsorbent of claim 1, wherein the binderless adsorbent has a BaO/(K₂O+BaO+Na₂O) molar ratio of from about 0.6 to about 0.85.

4. The adsorbent of claim 3, wherein the BaO/(K₂O+BaO+Na₂O) molar ratio is from about 0.65 to about 0.8.

5. The adsorbent of claim 1, wherein the binderless adsorbent has a Na₂O/(K₂O+BaO+Na₂O) molar ratio of from about 0.001 to about 0.04.

6. The adsorbent of claim 1, wherein the binderless adsorbent has a water content of from about 2 to about 5 wt. % of the binderless adsorbent.

7. The adsorbent of claim 1, wherein the binderless adsorbent has a silica to alumina molar ratio of from about 2 to about 2.6.

8. The adsorbent of claim 1, wherein the binderless adsorbent has a prepared portion of the zeolite X and a converted portion of the zeolite X, and wherein the prepared portion of the zeolite X has a first silica to alumina molar ratio of from about 2.4 to about 2.6.

9. The adsorbent of claim 8, wherein the converted portion of the zeolite X has a second silica to alumina molar ratio of from about 2 to about 2.2.

10. The adsorbent of claim 1, wherein the zeolite X comprises nano-size zeolite X.

11. The adsorbent of claim 10, wherein the binderless adsorbent has a prepared portion of the zeolite X and a converted portion of the zeolite X, and wherein the converted portion of the zeolite X comprises the nano-size zeolite X.

12. The adsorbent of claim 11, wherein the prepared portion of the zeolite X has an average crystalline size of from about 1 to about 3 μm.

13. A method for separating para-xylene from a mixture of C₈ alkyl aromatic hydrocarbons, the method comprising the steps of:
contacting a binderless adsorbent with a feed stream comprising the mixture of C₈ alkyl aromatic hydrocarbons to adsorb para-xylene from the feed stream and form a para-xylene-adsorbed binderless adsorbent, wherein the binderless adsorbent comprises zeolite X and has a K₂O/(K₂O+BaO+Na₂O) molar ratio of from about 0.15 to about 0.4; and
desorbing para-xylene from the para-xylene-adsorbed binderless adsorbent.

14. The method of claim 13, wherein the step of contacting comprises contacting the binderless adsorbent with the feed stream at a temperature of from about 100 to about 160° C.

15. The method of claim 13, wherein the step of desorbing comprises contacting the para-xylene-adsorbed binderless adsorbent with toluene to desorb para-xylene from the para-xylene-adsorbed binderless adsorbent and form an extract stream comprising toluene and para-xylene.

16. A method for making an adsorbent for separating para-xylene from a mixture of C₈ alkyl aromatic hydrocarbons, the method comprising the steps of:
forming a particle comprising a zeolite X precursor and a first portion of zeolite X;
activating the zeolite X precursor of the particle at activation conditions effective to form an activated zeolite X precursor;
digesting the particle with a caustic solution to convert the activated zeolite X precursor to a second portion of zeolite X and form a binderless zeolite X particle having ion exchangeable sites with cations; and
exchanging the cations with barium and potassium ions at the ion exchangeable sites to form a binderless adsorbent, wherein the binderless adsorbent comprises the zeolite X and has a K₂O/(K₂O+BaO+Na₂O) molar ratio of from about 0.15 to about 0.4.

17. The method of claim 16, wherein the step of activating comprises activating the zeolite X precursor at the activation conditions including a temperature of from about 500 to about 700° C.

18. The method of claim 16, wherein the step of digesting comprises digesting the particle with sodium hydroxide to convert the activated zeolite X precursor to the second portion of zeolite X.

19. The method of claim 16, wherein the step of exchanging the cations comprises ion exchanging the binderless zeolite X particle using a solution comprising potassium chloride and barium chloride.

20. The method of claim 16, wherein the step of exchanging the cations comprises forming the binderless adsorbent having the first portion of zeolite X present in an amount of from about 60 to about 95 wt. % of binderless adsorbent and the second portion of zeolite X present in an amount of from about 5 to about 40 wt. % of the binderless adsorbent.
